(19) United States
(12) Patent Application Publication
(10) Pub. No.: US 2020/0222680 A1
(43) Pub. Date: Jul. 16, 2020

(54) DEVICE AND METHOD FOR LOCKING AND TAGGING A MEDICAL TUBE

(71) Applicant: Kenneth SMITH, St. Paul, MN (US)

(72) Inventor: Kenneth SMITH, St. Paul, MN (US)

(21) Appl. No.: 16/338,851

(22) PCT Filed: Oct. 12, 2017
(86) PCT No.: PCT/US2017/056314

§ 371 Date: Apr. 2, 2019

Related U.S. Application Data
(60) Provisional application No. 62/407,225, filed on Oct. 12, 2016.

(51) Int. Cl. A61M 39/10; A61M 39/20

(52) U.S. Cl. CPC A61M 39/1011; A61M 2039/087; A61M 39/20

(57) ABSTRACT

Methods and devices for locking and tagging a medical tube are disclosed. The locking device connects to a medical tube and restricts full manipulation of the medical tube. The locking and tagging devices can communicate instructions or notices relevant to the medical tube, including its placement, purpose, function, operation, care, maintenance, removal, replacement, indication of manipulation or tampering, as well as the patient's healthcare information and health care provider information. In some configurations, the tagging device may be configured to communicate instructions and notices as a primary function, with or without a locking function.

### FIELD

This disclosure relates generally to medical tubes (also known as medical tubing), such as, for example, urinary catheters.

### BACKGROUND

Medical tubes, such as ports, drains, lines, and catheters, are used in the healthcare field for diagnostic and therapeutic purposes. For example, placement of an intravenous catheter may allow for infusion of radiographic agents for an imaging study, or for infusion of antibiotics to treat an infection. Another example of a medical tube could be the placement of a urinary catheter to drain urine from a patient.

### SUMMARY

Following the initial placement of a medical tube into a patient, the use of a medical tube typically follows a particular instruction set (e.g., specified protocol, patient needs, time limitation, special circumstances, etc.) for each patient. The particular instruction set with respect to the medical tube can be different for each patient. It is crucial that the medical tubing be used in accordance with the particular instruction set to reduce potential harm to the patient.

For example, after a urethral reconstruction surgery, the patient requires a duration of time for the affected tissues to heal. Accordingly, a urethral catheter may be placed into the patient for a duration of time in order for the affected tissues to heal. During this duration of time, the directive(s) for the patient's recovery may require that the urethral catheter not be manipulated (e.g., removed, cycled, flushed, replaced, etc.). If the urethral catheter is erroneously manipulated, unnecessary harm to the patient can occur.

Erroneous manipulation of a medical tube can be dangerous for the patient's health, increase cost of care, and may cause unnecessary complications such as psychological trauma, discomfort, pain, infection, bleeding, tissue damage, organ damage, and need for additional diagnostic studies, treatments, and procedures. Erroneous manipulation of a medical tube can be due to many reasons, such as a lack of knowledge, lack of understanding, or lack of adherence to instructions and notices relevant to the medical tube. Further, it has been discovered that when a medical tube is placed in a patient, a user (e.g., the patient, the patient's aide, or a health professional) may erroneously manipulate the medical tube.

Disclosed herein are devices and methods for preventing erroneous manipulation of a medical tube. Embodiments disclosed herein are directed towards devices that secure to a medical tube or lock one or more features of the medical tube. Some of the embodiments of the devices described herein are configured with a locking portion that connects to a medical tube and restricts full use of the medical tube, thereby preventing erroneous manipulation of the medical tube. By way of example, the device can attach to a portion of a medical tube to prevent the portion of the medical tube from being accessed. For instance, the device can cover an access port of the medical tube to prevent a syringe from being inserted into the access port, wherein the syringe is needed to deflate a balloon placed inside the patient for securing the medical tube to the patient.

In some embodiments, the device includes an information portion, either as part of the body of the device or as a part of a tag, which provides information to a user about instructions and notices relevant to the medical tube. The instructions and notices can include information relevant to a medical tube's insertion date, medical tube's expected removal date, medical tube's expected replacement date, medical tube's operational instructions, patient treatment protocol, doctor's contact information, a location of a patient chart, evidence of tampering or manipulation of the device or medical tube, or a combination thereof. The information portion of the device may communicate the instructions and notices visually (e.g., user notations or manipulations, text, graphics, barcode, color code, device appearance, illumination, etc.), palpably (e.g., shape, texture, vibration, surface features, etc.), non-visually (e.g., infrared notices, ultraviolet notices), auditorily, via signal technology (e.g., wireless link, radiofrequency, telemetry, other electromagnetic techniques, or an on-device memory module), or any combinations thereof. For example, the instructions and notices relevant to a medical tube may be communicated by the information portion of the device via handwritten marks, printed text, color pattern, tamper-evident features (e.g., mechanochromic pattern, material crazing and cracking, stress concentrations pattern, QR barcode, surface texture, audible alarm, RFID tag, readable memory module, or a combination thereof.

Some of the embodiments of the devices described herein are configured with a portion that connects to a medical tube and a portion that communicates instructions and notices. That is, some embodiments of the device may communicate instructions and notices without locking a particular feature of the medical tube

### BRIEF DESCRIPTION OF THE DRAWINGS

References are made to the accompanying drawings that form a part of this disclosure, and which illustrate the embodiments in which the devices and methods described herein can be practiced. Like references numbers indicate like parts throughout the disclosure.

FIG. 1 shows an environmental illustration of a medical tube with a locking device according to an embodiment.

FIG. 2 shows a schematic illustration of an embodiment of a locking device.

FIG. 3 shows a schematic illustration of another embodiment of a locking device.

FIG. 4A shows a schematic illustration of another embodiment of a locking device.

FIG. 4B shows a sectional side view of the embodiment of the locking device shown in FIG. 4A.

FIG. 5 shows a schematic illustration of another embodiment of a locking device.

FIG. 6 shows a schematic illustration of another embodiment of a locking device.

FIGS. 7A and 7B show schematic illustrations of an embodiment of a tagging device.

FIG. 8 shows a schematic illustration of an embodiment of a tagging device.

FIG. 9 shows a schematic illustration of an embodiment of a tagging device.

### DETAILED DESCRIPTION

Embodiments described herein relate to a device for use with a medical tube. More specifically, embodiments described herein are directed toward a device which includes a body portion for connecting to a medical tube and a locking portion for restricting full use of the medical tube. An aspect of the device includes an information portion for communicating instructions and notices relevant to the medical tube. Examples of the medical tube include, but are not limited to, externalized drains, such as for example, HemoVac drain, penrose drain, Blake drain, wick drain, Jackson Pratt drain, suction drain, potential space drain, weep drain, wound vacuum drain, thoracic drain, abdominal drain, peritoneal drain, abscess drain, lymph drain, chyle drain, urinoma drain, hematoma drain, fistula drain, joint space drain, and wound drain. Other examples of the medical tube include, but are not limited to, ventriculostomy tube, nasal tube, oral tube, ear canal tube, endotracheal tube, tracheostomy tube, gastrostomy tube, jejunostomy tube, nasogastric tube, nasojejunal tube, orogastric tube, orojejunal tube, feeding tube, cecostomy tube, enterocutaneous tube, hepato-pancreatico-biliary tube, rectal tube, and Sengstaken-Blakemore tube. Additional examples of the medical tube include, but are not limited to, intraarterial and intravenous lines, such as for example, peripheral intravenous line, femoral line, peripherally inserted central catheter (PICC), femoral sheath, cannula, midline central catheter, umbilical catheter, central intravenous line, intraaortic balloon pump lines, Swan-Ganz line, left ventricular assist device lines, arterial line, and percutaneous dialysis catheter. Other examples of the medical tube include, but are not limited to, epidural catheter, intradural catheter, subarachnoid catheter, intrathecal catheter, subcutaneous catheter, suprapubic catheter, appendicovesicostomy catheter, urethral catheter, urethrostomy catheter, ureteral stent, urostomy catheter, condom catheter, ureteral catheter, nephrostomy tube, regional anesthesia catheter, vaginal catheter, intrauterine catheter, chest tube, thoracostomy tube, pericardial tube, stoma cannula, ostium tube, tissue expander line, intraparenchymal tube, intramuscular tube, intraosseous tube, joint space tube, intraarticular tube, potential space tube, compartment space tube, and intermittent or continuous irrigation tube.

With regard to the foregoing description, the term medical tube can include one or more element(s) or component(s) that is(are) connected to or connectable to the medical tube itself. That is, the term medical tube as used herein can include one or more of, for example, stopcock, valve, connector, filter, meter, clamp, port, manifold, vent, sensor, sheath, shroud, occluder, reservoir, drain, fitting, conditioner, separator, mount, control element, regulator, advancer, syringe, dilater, bulb, indicator, housing, plunger, cover, extender, channel, container, regulator, shaft, nub, needle, spike, adapter, cap, flange, cannula, obturator, aperture, chamber, cuff, etc. Accordingly, a device which connects to a medical tube can include a device which connects to an element or component which connects to a medical tube.

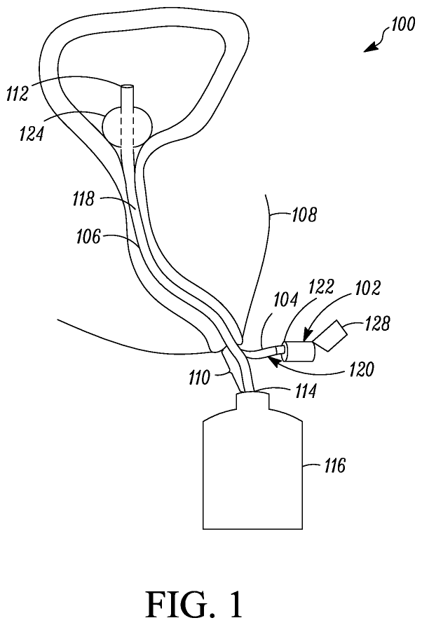

FIG. 1 shows an exemplary environment 100 for an embodiment of a locking device 102 which is connected to a medical tube 104 (for example, but not limited to, a urinary catheter). A portion 106 of the medical tube 104 is placed within a patient 108 (e.g., urethra and bladder). Another portion 110 of the medical tube is kept outside the patient 108. The portion 106 of the medical tube 104 to be placed within the patient 108 shown in this example includes a bladder opening 112 which is positioned to be inside the bladder of the patient 108. The portion 110 of the medical tube 104 placed outside the patient 108 includes a collecting end 114 connected to a urine collecting bag 116. The bladder opening 112 and the collecting end 114 are connected via a urine drain tube 118.

The medical tube 104 includes another tube 120 which is hydraulically isolated from the urine drain tube 118. The urine drain tube 118 and the tube 120 forming an upside-down Y shape as shown in FIG. 1. The tube 120 includes a balloon port 122 at one end, which is an opening for the tube 120 to have access to an inflatable balloon 124. The inflatable balloon 124 is disposed in the portion 106 of the medical tube 104 that is to be placed within the patient 108. More specifically, the inflatable balloon 124 is configured to be inside the bladder of the patient 108. The inflatable balloon 124 is placed in the bladder before it is inflated, and then when placed within the bladder, the inflatable balloon 124 is inflated via injection of a liquid (e.g., sterile water) using a syringe (not shown) via the balloon port 122. The syringe is generally removed from the balloon port 122 after it is used to deliver the liquid to inflate the balloon 124. When the removal of the medical tube 104 is required, the syringe can be connected to the balloon port 122 to withdraw the fluid from the balloon 124, which causes the balloon 124 to deflate. The balloon 124 in the deflated state allows the medical tube 104 to be pulled out of the bladder and through the urethra of the patient 108.

FIG. 1 shows the inflatable balloon 124 in the inflated state, positioned in the bladder of the patient 108. The inflated balloon 124 stabilizes the position of the medical tube 104 so that the urine can flow through the urine drain tube 118 from the bladder opening 112 to the urine collecting bag 116. To prevent erroneous manipulation of the balloon port 122, the locking device 102 is connected to the balloon port 122. Removal of the locking device 102 is required to access the balloon port 122, for example to deflate the balloon 124 for removal of the medical tube 104 from the patient 108. Thus, erroneous removal of the medical tube 104 from the patient 108 can be prevented by having the locking device 102 connected to the balloon port 122.

In an embodiment, the locking device 102 can be connected to the balloon port 122 in a locking manner after the balloon 124 has been inflated, so that the locking device 102 must be destroyed in order to remove the locking device 102 from the balloon port 122. Thus, erroneous removal of the medical tube 104 from the patient 108 can be prevented by having the locking device 102 locked on to the balloon port 122. Furthermore, destructive removal of the locking device 102 from medical tube 104 could serve as an indication that the medical tube 104 had been manipulated. Furthermore, destructive or non-destructive removal of the locking device 102 from medical tube 104 could be indicated by tamper-evident features, such as, for example, changes in visual appearance, physical configuration, or material properties of locking device 102 when the locking device 102 is manipulated.

The locking device 102 can include a tag 128 for communicating information. In an embodiment, the information communicated with the tag 128 includes instructions and notices relevant to the medical tube 104. In another embodiment, the body of locking device 102 can communicate instructions and notices relevant to the medical tube 104.

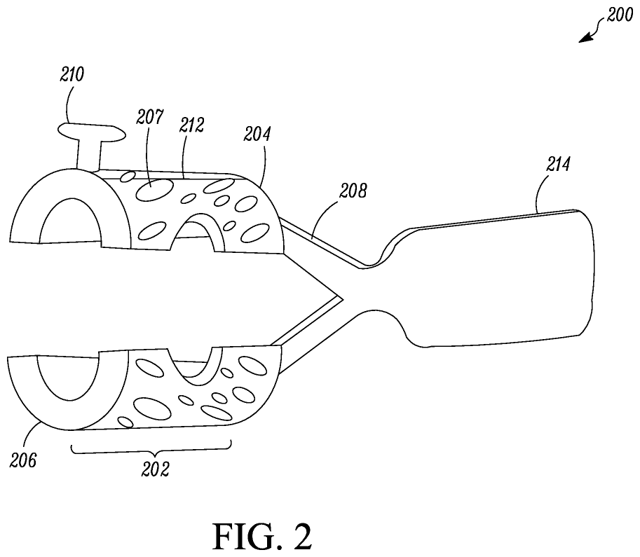

FIG. 2 shows a schematic illustration of an embodiment of a locking device 200. The locking device 200 can attach to a balloon port (e.g., 122 shown in FIG. 1) of a medical tube (e.g., 104 shown in FIG. 1).

The locking device 200 includes a main body portion 202 made of two parts 204, 206 connected via a hinge portion 208. The hinge portion 208 is flexible to allow the two parts 204, 206 to come together and be joined around the balloon port to connect the locking device 200 to the balloon port. The two parts 204, 206 are fashioned with open passages 207 (e.g., fenestrations) to preclude a closed space when the two parts 204, 206 are in contact. The two parts 204, 206 have respective mating surfaces that connect together, such that removal of the locking device 200 from the balloon port would require separation of the two parts 204, 206 using some amount of force. Alternatively, once the two parts 204, 206 are joined at the balloon port, a destruction of the locking device 200 is required for removal of the locking device 200 from the balloon port.

The two parts 204, 206 can also have notches such that the locking device 200 can be secured onto the shaft of the medical tube in such an orientation where the long axis of the locking device 200 could be perpendicular to the long axis of the medical tube. In such a configuration, the locking device 200 could tag the medical tube and communicate instructions and notices, without necessarily impeding a user's full access to the medical tube.

The respective mating surfaces of the two parts 204, 206 can be smooth, rough, abrasive, tacky, chemically-treated, electromagnetically-treated, or combinations thereof to form a connection to the medical tube when the mating surfaces of two parts 204, 206 contact each other. For example, a smooth surface can include a sealant that is comprised of a glue, adhesive, or sealing component. For example, an abrasive surface can have nodules to interlock when contact is made. For example, a chemically-treated surface could form a thermoplastic bond when contact is made. Alternatively, the locking device 200 could have two parts 204, 206 with protuberances that mechanically interdigitate to secure the locking device 200 onto the medical tube.

Alternatively, the locking device 200 could be embodied in a neutral configuration such that the two parts 204, 206 are in close proximity to each other (e.g., closed position), and some amount of force would be required to distract the two parts 204, 206 into an open configuration, such that the locking device 200 could be connected to the medical tube. Once the locking device 200 is positioned at the medical tube, subsequent removal of the distracting force from the two parts 204, 206 then would result in the locking device returning toward its neutral stress state (e.g., closed position), thus providing a connection with the medical tube.

The locking device 200 includes a removal tab 210 at one of the parts 204 of the body portion 202. The removal tab 210 is accompanied by one or more scoring 212 (e.g., laser scoring) near the removal tab 210. The scoring 212 tears in a predetermined manner along the scoring 212 when the removal tab 210 is pulled, resulting in the destruction of the part 204 of the body portion 202. The destruction of the part 204 allows the locking device 200 to be removed from the balloon port easily.

The destruction of the part 204 prevents the locking device 200 from being used again to connect to the balloon port. As such, a single-use configuration of locking device 200 could serve as a deterrent to a user's manipulation the medical tube, the locking device 200, or both the medical tube and locking device 200.

Furthermore, manipulation of removal tab 210 provides an indication of tampering of the locking device 200. In some embodiments, any manipulation (destructive or non-destructive) of the removal tab 210 and/or locking device 200, once connected to the medical tube, results in an overt and/or covert distortion of the device as a tamper-evident feature. Other tamper-evident features can be incorporated into the locking device 200; for example, physical stress applied to the removal tab 210, main body portion 202, the two parts 204, 206, hinge portion 208, or tag 214 could result in material crazing, cracking, fracture, or rupture of locking device 200 as visible evidence to a user that tampering has occurred. Furthermore, tamper-evident features of locking device 200 could include, for example, overt and/or covert notices employing visual and non-visual displays, auditory alerts, vibratory alerts, surface texture changes, surface optical changes, illumination property changes, wireless link notifications, on-device memory state changes, electronic data interchanges, machine-readable notifications, or any combination thereof.

The locking device 200 includes a tag 214 for communicating information. The information communicated with tag 214 can include instructions and notices relevant to the medical tube 104. Instructions and notices relevant to the medical tube 104 could also be communicated with the main body portion 202, the two parts 204, 206, or hinge portion 208.

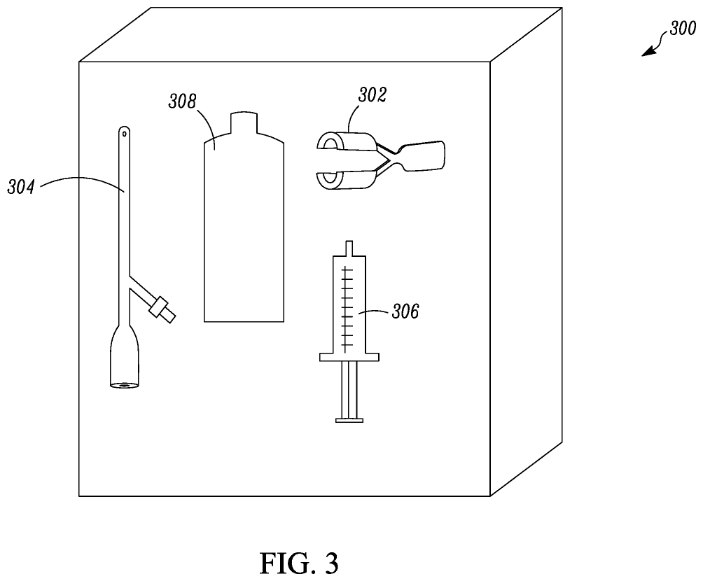

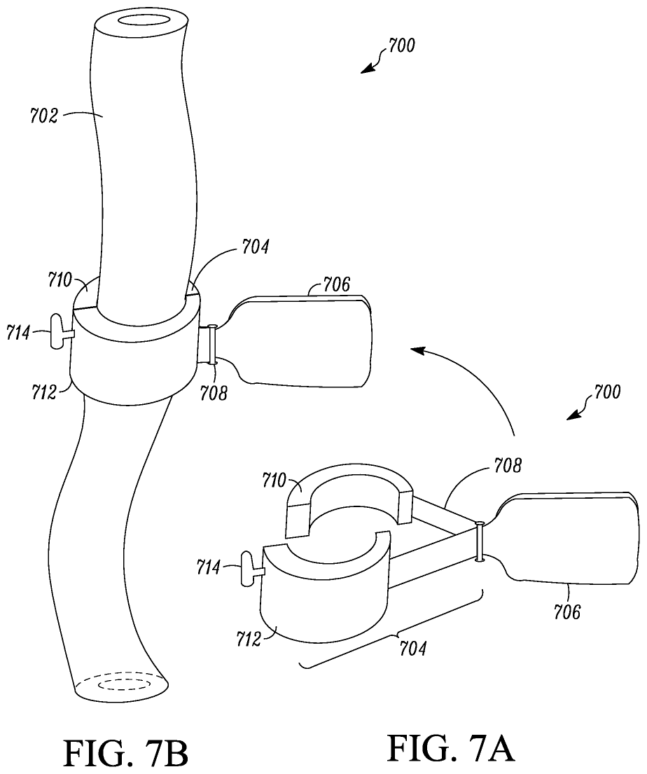

FIG. 3 shows an exemplary medical tube kit, which is a catheter kit 300 containing, at least, a locking device 302 (which can be a tagging device), a catheter 304 having a Y-shape formed by a urine drain tube and a balloon inflating tube, a syringe 306 filled with a fluid for inflating the balloon, and a waste collection bag 308. A user can open the catheter kit 300, insert the catheter 304 into the patient (e.g., as shown in FIG. 1), take the syringe 306 out from the catheter kit 300, connect the syringe 306 to the catheter 304 at a balloon port (e.g., 122 shown in FIG. 1) of the catheter 304, and deliver the fluid from the syringe 306 to the balloon and inflate the balloon. The inflated balloon secures the catheter 304 to the patient, as the balloon should be inflated inside the patient's bladder. Then, the syringe is disconnected from the balloon port of the catheter 304. The locking device 302 is secured to, connected to, or locked onto the balloon port of the catheter 304, which prevents removal of the fluid from the balloon via the balloon port. That is, the user can be prevented from accessing the balloon port of the catheter 304 while the locking device 302 is connected to the catheter 304. Further, the user can refer to the body or tag portion of locking device 302 to confirm instructions and notices relevant to the catheter 304 prior to manipulating the catheter 304, such as removing the locking device 302. Hence, once connected to the catheter 304, the locking device 302 can prevent erroneous manipulation of catheter 304 (e.g., cycling of the inflatable balloon, removal of catheter 304, replacement of catheter 304, etc.). If a tagging device (e.g., as shown in FIGS. 7A, 7B, 8, and 9) is included in the kit 300, the tagging device can be secured to a tube portion of the medical tube, instead of being connected to the balloon port of the catheter 304.

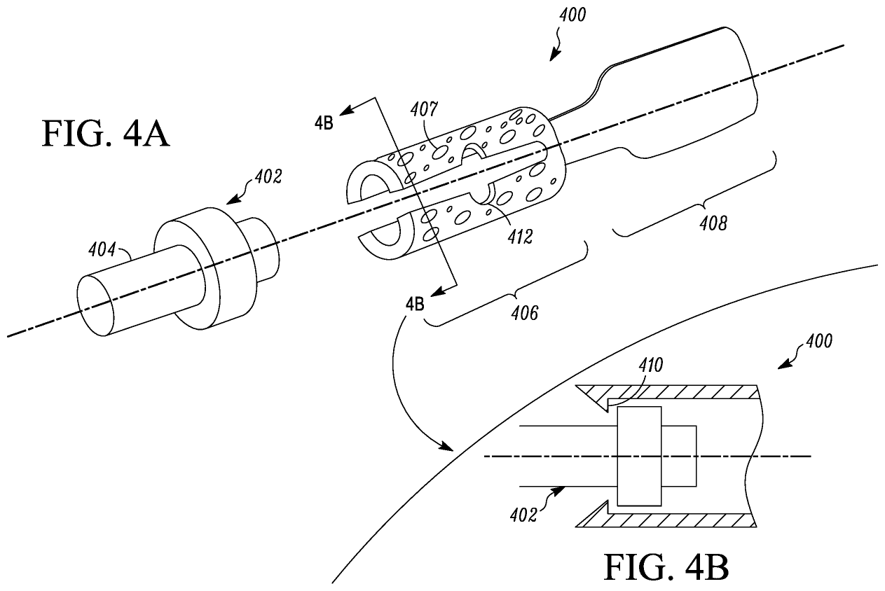

FIG. 4A shows an embodiment of a locking device 400 which is configured to connect to a terminus 402 of a medical tube 404 by being pushed onto the terminus 402. FIG. 4B shows an enlarged sectional side view of the locking device 400 and the terminus 402. The locking device 400 includes a body portion 406 and a tag 408. The tag 408 is for communicating information. The information communicated with tag 408 can include instructions and notices relevant to the medical tube 404. Instructions and notices relevant to the medical tube 404 could also be communicated with the body portion 406. The body portion 406 has open passages 407 (e.g., fenestrations) to preclude a closed space when the body portion 406 is secured onto the terminus 402. The locking device 400 has one or more engagement portions 410 for securing to a region near the terminus 402 without requiring a formal hinge mechanism (e.g., hinge portion 208 of the embodiment 200 shown in FIG. 2). Rather than requiring the hinge mechanism, the locking device 400 is made of a material which can deform during engagement (e.g., being pushed) to the terminus 402. The material allows for the locking device 400 to return toward its neutral stress state once in position at the terminus 402. Alternatively, the locking device 400 is configured to exert stress on to the terminus 402 to secure the locking device onto the terminus 402. The body portion 406 of the locking device 400 can have an open region 412 for being secured onto a tube portion (e.g., not the terminus 402) of the medical tube 404. Accordingly, the locking device 400 can function as a tagging device for the medical tube 404 without necessarily preventing access to the terminus 402.

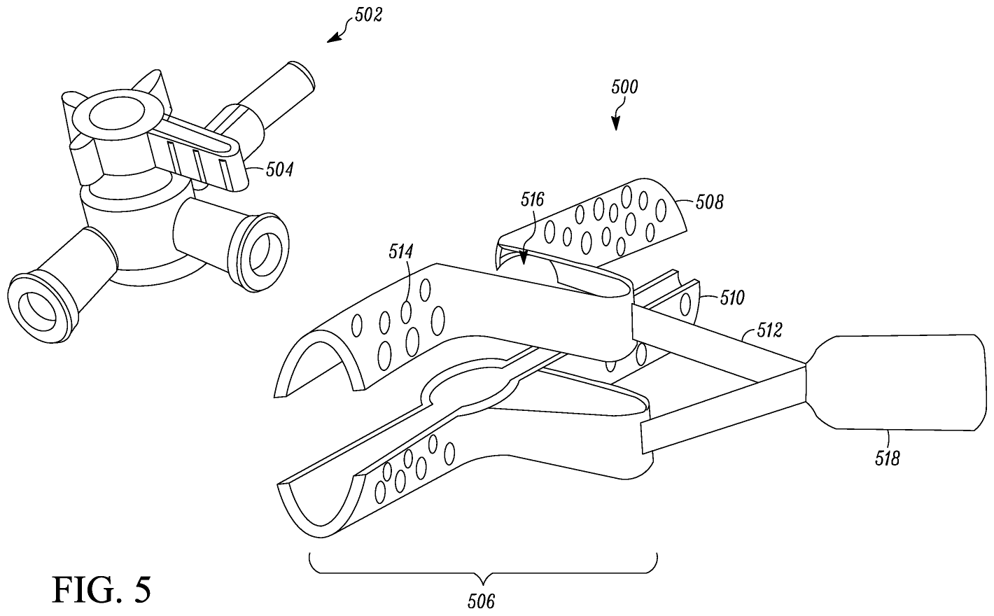

FIG. 5 shows an embodiment of a locking device 500. The locking device 500 can attach to a three-way stopcock 502 (a mechanically operated valve regulating the flow of a fluid) of a medical tube(s). The handle 504 of the three-way stopcock 502 controls the direction of the fluid flow. The locking device 500 is configured to connect to the three-way stopcock 502 to prevent manipulation of the handle 504 while the locking device 500 is connected to the three-way stopcock 502.

The locking device 500 includes a main body portion 506 made of two parts 508, 510 connected via a hinge portion 512. The hinge portion 512 is flexible to allow the two parts 508, 510 to come together and be joined around the three-way stopcock 502 to connect the locking device 500 to the three-way stopcock 502. The two parts 508, 510 are fashioned with open passages 514 (e.g., fenestrations) to preclude a closed space when the two parts 508, 510 are in contact. The two parts 508, 510 have respective mating surfaces that connect together, such that removal of the locking device 500 from the three-way stopcock 502 would require separation of the two parts 508, 510 using some amount of force. Alternatively, once the two parts 508, 510 are joined at the three-way stopcock 502, a destruction of the locking device 500 is required for removal of the locking device 500 from the three-way stopcock 502.

At least one of the two parts 508, 510 has a hole 516 configuration for receiving the handle 504 of the three-way stopcock 502 when the two parts 508, 510 come together to be joined around the three-way stopcock 502. Thus, while the locking device 500 is connected to the three-way stopcock 502, mechanical operation of the handle 504 to change fluid flow in the three-way stopcock 502 can be prevented.

The respective mating surfaces of the two parts 508, 510 can be smooth, rough, abrasive, tacky, chemically-treated, electromagnetically-treated, or combinations thereof to form a connection to the three-way stopcock 502 when the mating surfaces of two parts 508, 510 contact each other. For example, a smooth surface can include a sealant that is comprised of a glue, adhesive, or sealing component. For example, an abrasive surface can have nodules to interlock when contact is made. For example, a chemically-treated surface could form a thermoplastic bond when contact is made. Alternatively, the locking device 500 could have two parts 508, 510 with protuberances that mechanically interdigitate to secure the locking device 500 onto the three-way stopcock 502.

Alternatively, the locking device 500 could be embodied in a neutral configuration such that the two parts 508, 510 are in close proximity to each other (e.g., closed position), and some amount of force would be required to distract the two parts 508, 510 into an open configuration, such that the locking device 500 could be connected to the three-way stopcock 502. Once the locking device 500 is positioned at the three-way stopcock 502, subsequent removal of the distracting force from the two parts 508, 510 then would result in the locking device returning toward its neutral configuration (e.g., closed position), thus providing a connection with the three-way stopcock 502.

The locking device 500 includes a tag 518 for communicating information. The information communicated with tag 518 can include instructions and notices relevant to the three-way stopcock 502 or the medical tube. Instructions and notices relevant to the three-way stopcock 502 or the medical tube could also be communicated with the main body portion 506, the two parts 508, 510, or hinge portion 512.

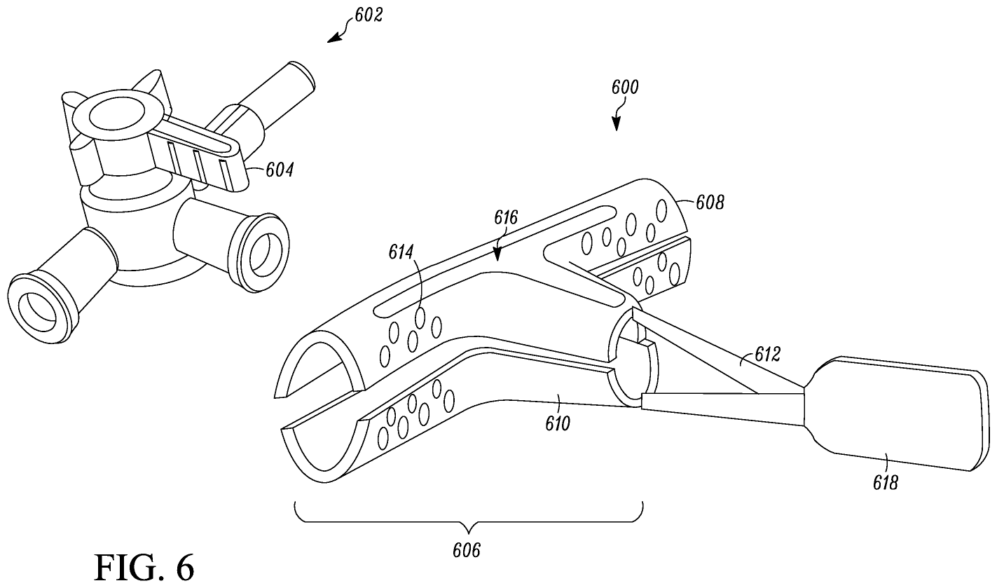

FIG. 6 shows another embodiment of a locking device 600. The locking device 600 can attach to a three-way stopcock 602 (a mechanically operated valve regulating the flow of a fluid) of a medical tube(s). The handle 604 of the three-way stopcock 602 controls the direction of the fluid flow. The locking device 600 is configured to connect to the three-way stopcock 602 to prevent manipulation of the handle 604 while the locking device 600 is connected to the three-way stopcock 602.

The locking device 600 includes a main body portion 606 made of two parts 608, 610 connected via a hinge portion 612. The hinge portion 612 is flexible to allow the two parts 608, 610 to come together and be joined around the three-way stopcock 602 to connect the locking device 600 to the three-way stopcock 602. The two parts 608, 610 are fashioned with open passages 614 (e.g., fenestrations) to preclude a closed space when the two parts 608, 610 are in contact. The two parts 608, 610 have respective mating surfaces that connect together, such that removal of the locking device 600 from the three-way stopcock 602 would require separation of the two parts 608, 610 using some amount of force. Alternatively, once the two parts 608, 610 are joined at the three-way stopcock 602, a destruction of the locking device 600 is required for removal of the locking device 600 from the three-way stopcock 602.

At least one of the two parts 608, 610 has a hole 616 configuration for receiving the handle 604 of the three-way stopcock 602 when the two parts 608, 610 come together to be joined around the three-way stopcock 602. The hole 616 is shaped to be able to receive the handle 604 in any of its operational/nonoperational positions. Thus, while the locking device 600 is connected to the three-way stopcock 602, manipulation of the handle 604 to change fluid flow in the three-way stopcock 602 can be prevented.

The respective mating surfaces of the two parts 608, 610 can be smooth, rough, abrasive, tacky, chemically-treated, electromagnetically-treated, or combinations thereof to form a connection to the three-way stopcock 602 when the mating surfaces of two parts 608, 610 contact each other. For example, a smooth surface can include a sealant that is comprised of a glue, adhesive, or sealing component. For example, an abrasive surface can have nodules to interlock when contact is made. For example, a chemically-treated surface could form a thermoplastic bond when contact is made. Alternatively, the locking device 600 could have two parts 608, 610 with protuberances that mechanically interdigitate to secure the locking device 600 onto the three-way stopcock 602.

Alternatively, the locking device 600 could be embodied in a neutral configuration such that the two parts 608, 610 are in close proximity to each other (e.g., closed position), and some amount of force would be required to distract the two parts 608, 610 into an open configuration, such that the locking device 600 could be connected to the three-way stopcock 602. Once the locking device 600 is positioned at the three-way stopcock 602, subsequent removal of the distracting force from the two parts 608, 610 then would result in the locking device resuming a configuration toward its neutral stress state (e.g., closed position), thus providing a connection with the three-way stopcock 602.

The locking device 600 includes a tag 618 for communicating information. The information communicated with tag 618 can include instructions and notices relevant to the three-way stopcock 602 or the medical tube. Instructions and notices relevant to the three-way stopcock 602 or the medical tube could also be communicated with the main body portion 606, the two parts 608, 610, or hinge portion 612.

FIGS. 7A and 7B show an embodiment of a tagging device 700 configured to connect to a medical tube 702. FIG. 7A shows the tagging device 700 prior to being connected to the medical tube 702. FIG. 7B shows the tagging device 700 connected to the medical tube 702. The tagging device 700 includes a body portion 704 and a tag 706. The tag 706 is for communicating information. The information communicated with tag 706 can include instructions and notices relevant to the medical tube 702. Instructions and notices relevant to the medical tube 702 could also be communicated with the body portion 704. The body portion 704 is configured for securing to the medical tube 702. The body portion 704 includes a hinge portion 708. The hinge portion 708 is flexible to allow the two parts 710, 712 to come together and be joined around the medical tube 702. The two parts 710, 712 can have respective mating surfaces that connect together, such that removal of the tagging device 700 from the medical tube 702 would require separation of the two parts 710, 712 using some amount of force. Alternatively, a destruction of the tagging device 700 is required for removal. The respective mating surfaces of the two parts 710, 712 can be smooth, rough, abrasive, tacky, chemically-treated, electromagnetically-treated, or combinations thereof to form a connection to the medical tube 702 when the mating surfaces of two parts 710, 712 contact each other. For example, a smooth surface can include a sealant that is comprised of a glue, adhesive, or sealing component. For example, an abrasive surface can have nodules to interlock when contact is made. For example, a chemically-treated surface could form a thermoplastic bond when contact is made. Alternatively, the two parts 710, 712 are in close proximity to each other (e.g., closed position), and some amount of force would be required to distract the two parts 710, 712 into an open configuration, such that the tagging device 700 could be connected to the medical tube 702. Once the tagging device 700 is positioned at the medical tube 702, subsequent removal of the distracting force from parts 710, 712 then would result in the tagging device 700 resuming a configuration toward its neutral stress state (e.g., closed position), thus providing a connection with the medical tube 702. The tagging device 700 includes a removal tab 714 at one of the parts of the body portion 704. The removal tab 714 can be accompanied by one or more scoring (e.g., laser scoring) near the removal tab 714. The scoring tears in a predetermined manner along the scoring when the removal tab 714 is pulled, resulting in the destruction of one or more parts 710, 712 of the body portion 704.

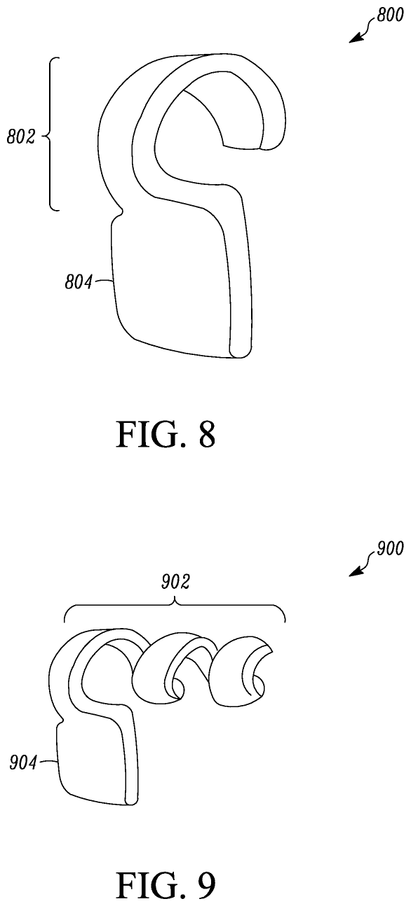

FIG. 8 shows another embodiment of a tagging device 800 configured to connect to a medical tube. The tagging device 800 includes a body portion 802 and a tag 804. The tag 804 is for communicating information, similarly to the tag 706 shown in FIGS. 7A and 7B. Information could also be communicated with the body portion 802. The body portion 802 has a loop shape configured for securing to the medical tube without requiring a formal hinge mechanism (e.g., hinge portion 708 shown in FIGS. 7A and 7B). Rather than requiring the hinge mechanism, the tagging device 800 can be made of a material which can deform, at least partly, during engagement (e.g., being pushed) to the medical tube. The material of the body portion 802 allows for the tagging device 800 to return toward its neutral stress state once in position connected to the medical tube.

FIG. 9 shows yet another embodiment of a tagging device 900 configured to connect to a medical tube. The tagging device 900 includes a body portion 902 and a tag 904. The tag 904 is for communicating information, similarly to the tag 706 shown in FIGS. 7A and 7B. Information could also be communicated with the body portion 902. The body portion 902 has a helical shape configured for looping around the outer surface of the medical tube such that the body portion 902 can be secured to the medical tube. The tagging device 900 can be made of or include a material which can deform or is flexible, so that the tagging device 900 can be wrapped around the medical tube.

Alternatively, the tagging device 900 can be made of a material which cannot easily deform. That is, the tagging device 900 can be made of a stiff material, and the medical tube can be looped through the body portion 902 without requiring the body portion 902 to deform or flex.

Aspects:

Any of the following aspects can be combined with any one or more of the other aspects.

Aspect 1. A device for connecting to a medical tube, comprising:

a body portion; and

a locking portion for connecting to the medical tube

Aspect 2. The device according to aspect 1, wherein the locking portion prevents full access or use of the medical tube when connected to the medical tube.
Aspect 3. The device according to any of aspects 1 and 2, further comprising an information portion.
Aspect 4. The device according to any of aspects 1-3, wherein the information portion includes a visually displayed notice.
Aspect 5. The device according to any of aspects 1-4, wherein the information portion includes a non-visually displayed notice.
Aspect 6. The device according to any of aspects 1-5, wherein the non-visually displayed notice includes one or more of palpable notice, and auditory notice.
Aspect 7. The device according to any of aspects 1-6, wherein the information portion includes a wirelessly transmitted notice.
Aspect 8. The device according to any of aspects 1-7, wherein the wirelessly transmitted notice is a transmitted signal by one or more of WiFi, Bluetooth, radio frequency (RFID), telemetry, and electromagnetic field.
Aspect 9. The device according to any of aspects 1-8, wherein the information portion includes an internet address.
Aspect 10. The device according to any of aspects 1-9, wherein the information portion includes one or more of one-dimensional barcode, and multi-dimensional barcode.
Aspect 11. The device according to any of aspects 1-10, wherein the information portion includes data regarding the medical tube, wherein the data includes one or more of placement detail, purpose, function, general condition, operation, care, maintenance, removal, and replacement.
Aspect 12. The device according to any of aspects 1-11, wherein the information portion includes data regarding the medical tube, wherein the data includes one or more of responsible health care provider's name, responsible health care provider's contact information, location of patient chart, and location of additional instruction.
Aspect 13. The device according to any of aspects 1-12, further comprising a hinge connected to the body portion, wherein the body portion comprises a first part and a second part, wherein the first part and the second part can connect at respective mating surfaces.
Aspect 14. The device according to any of aspects 1-13, wherein the locking portion is made of a deformable material, wherein the locking portion can deform when connecting to the medical tube.
Aspect 15. The device according to any of aspects 1-14 that does not include a hinge connected to the body portion.
Aspect 16. The device according to any of aspects 1-15, wherein the locking portion includes one or more engagement portion(s) for locking the device onto an end portion of the medical tube.
Aspect 17. The device according to any of aspects 1-16, wherein the locking portion covers a branch of a medical tube and prevents a fluidic device from being hydraulically connected to the branch.
Aspect 18. The device according to any of aspects 1-17, wherein the locking portion engages a mechanical component of the medical tube and prevents manipulation of that mechanical component.
Aspect 19. The device according to any of aspects 1-18, wherein the locking portion has a cylindrical shape or a box shape when in a closed position.
Aspect 20. The device according to any of aspects 1-19, wherein the locking portion is a physical barrier to manipulating the medical tube.
Aspect 21. The device according to any of aspects 1-20, further comprising a removal tab, wherein the removal tab requires actuation for disconnecting the locking device from the medical tube.
Aspect 22. The device according to any of aspects 1-21, wherein the actuation of the removal tab destroys at least one of the body portion and the locking portion.
Aspect 23. The device according to any of aspects 1-22, further comprising a removal tab connected to a perforated edge on an axis of at least one of the body portion and the locking portion.
Aspect 24. The device according to any of aspects 1-23, wherein the removal tab is configured to destructively disconnect the locking device along the perforated edge.
Aspect 25. A medical tube kit, comprising:

a medical tube that includes a first end and a second end;

a y-joint at the first end of the medical tube, wherein the y-joint includes a first branch and a second branch;

an inflatable chamber at the second end of the medical tube;

a syringe to inflate the chamber via a first branch of the y-joint; a collection bag connected to the second branch of the y-joint; and

the locking device according to any of aspects 1-24, wherein the locking device attaches to the first branch to prevent full access to the first branch.

Aspect 26. A method of using a medical tube, comprising:

connecting a fluidic device to the medical tube;

operating the fluidic device in physical connection with the medical tube;

disconnecting the fluidic device from the medical tube; and

connecting the device according to any of aspects 1-24 to the medical tube.

Aspect 27. A method of using a medical tube, comprising:

operating a mechanical feature of the medical tube;

connecting the device according to any of aspects 1-24 to the medical tube.

Aspect 28. A device for connecting to a medical tube, comprising:

a body portion configured to connect to the medical tube; and

an information portion.

Aspect 29. The device according to aspect 28, wherein the information portion includes a visually displayed notice.

Aspect 30. The device according to any of aspects 28-29, wherein the information portion includes a non-visually displayed notice.
Aspect 31. The device according to any of aspects 28-30, wherein the non-visually displayed notice includes one or more of palpable notice, and auditory notice.
Aspect 32. The device according to any of aspects 28-31, wherein the information portion includes a wirelessly transmitted notice.
Aspect 33. The device according to any of aspects 28-32, wherein the wirelessly transmitted notice is a transmitted signal by one or more of WiFi, Bluetooth, radio frequency (RFID), telemetry, and electromagnetic field.
Aspect 34. The device according to any of aspects 28-33, wherein the information portion includes an internet address.
Aspect 35. The device according to any of aspects 28-34, wherein the information portion includes one or more of one-dimensional barcode, and multi-dimensional barcode.
Aspect 36. The device according to any of aspects 28-35, wherein the information portion includes data regarding the medical tube, wherein the data includes one or more of placement detail, purpose, function, general condition, operation, care, maintenance, removal, and replacement.
Aspect 37. The device according to any of aspects 28-36, wherein the information portion includes data regarding the medical tube, wherein the data includes one or more of responsible health care provider's name, responsible health care provider's contact information, location of patient chart, and location of additional instruction.
Aspect 38. The device according to any of aspects 28-37, further comprising a hinge connected to the body portion, wherein the body portion comprises a first part and a second part, wherein the first part and the second part can connect at respective mating surfaces.
Aspect 39. The device according to any of aspects 28-38, wherein the body portion is made of a deformable material, wherein the body portion can deform when connecting to the medical tube.
Aspect 40. The device according to any of aspects 28-39 that does not include a hinge connected to the body portion.
Aspect 41. A method of using a medical tube, comprising:

connecting a fluidic device to the medical tube;

operating the fluidic device in physical connection with the medical tube;

disconnecting the fluidic device from the medical tube; and

connecting the device according to any of aspects 28-40 to the medical tube.

Aspect 42. A method of using a medical tube, comprising:

operating a mechanical feature of the medical tube;

connecting the device according to any of aspects 28-40 to the medical tube.

Aspect 43. The device according to any of aspects 1-24, wherein the device is configured to include tamper-evident features.
Aspect 44. The device according to any of aspects 28-40, wherein the device is configured to include tamper-evident features.

With regard to the foregoing description, it is to be understood that changes may be made in detail, without departing from the scope of the patent invention. It is intended that the depicted embodiments are to be considered exemplary, with the scope of the invention being indicated by the broad meaning of the claims.

What is claimed is:

1. A device for connecting to a medical tube, comprising:
a body portion configured to connect to the medical tube; and
a locking portion configured to preclude full use of the medical tube when connected to the medical tube.

2. The device of claim 1, wherein the locking portion includes one or more engagement portion(s) to preclude full use of the medical tube when connected to the medical tube.

3. The device of claim 1, wherein the locking portion is configured to connect to one or more portions of the medical tube and precludes another device from being connected to one or more portions of the medical tube when connected to the medical tube.

4. The device of claim 1, wherein the locking portion is configured to engage a branch of the medical tube to preclude a fluidic device from being hydraulically connected to the branch when connected to the medical tube.

5. The device of claim 1, wherein the locking portion is configured to connect to one or more components of the medical tube and precludes full use of that(those) component(s) when connected to the medical tube.

6. The device of claim 1, wherein the device is configured to be distorted when the device is manipulated after being connected to the medical tube.

7. The device of claim 1, wherein the device is configured to convey a notice of manipulation of the device when the device is manipulated after being connected to the medical tube.

8. The device of claim 1, further comprising an information portion, wherein the information portion is configured to convey information relevant to the medical tube using at least one of a visual display, a non-visual display, an auditory alert, a tactile feature, a wireless link, an electromagnetic notice, a machine readable notice, or an electronic data interchange.

9. The device of claim 8, wherein the device is configured to be distorted when the device is manipulated after being connected to the medical tube.

10. The device of claim 8, wherein the device is configured to convey a notice of manipulation of the device when the device is manipulated after being connected to the medical tube.

11. A device for connecting to a medical tube, comprising:
a body portion configured to connect to the medical tube; and
an information portion, wherein the information portion conveys information relevant to the medical tube using at least one of a visual display, a non-visual display, an auditory alert, a tactile feature, a wireless link, an electromagnetic notice, a machine readable notice, or an electronic data interchange.

12. The device of claim 11, wherein the body portion includes one or more engagement portion(s) configured for connecting to the medical tube.

13. The device of claim 11, wherein the device is configured to be distorted when the device is manipulated after being connected to the medical tube.

14. The device of claim 11, wherein the device is configured to convey a notice of manipulation of the device when the device is manipulated after being connected to the medical tube.

15. A method of using a medical tube, comprising:
using the medical tube;
connecting the device according to claim 1 to the medical tube.

16. A method of using a medical tube, comprising:
using the medical tube;
connecting the device according to claim 8 to the medical tube.

17. A method of using a medical tube, comprising:
using the medical tube;
connecting the device according to claim 11 to the medical tube.
